(19) United States
(12) Patent Application Publication
(10) Pub. No.: US 2008/0138812 A1
(43) Pub. Date: Jun. 12, 2008

(54) Screening Method For Competitive Hiv Rt Inhibitors

(76) Inventors: Dirk Edward Desire Jochmans, Herent (BE); Piet Tom Bert Paul Wigerinck, Terhagen (BE)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 11/814,841

(22) PCT Filed: Feb. 6, 2006
(86) PCT No.: PCT/EP06/50708

§ 371 Date: Jul. 26, 2007

(30) Foreign Application Priority Data
Feb. 4, 2005 (EP) 05100811.8
Jun. 1, 2005 (EP) 05104766.0

(51) Int. Cl. C12Q 1/68; C12Q 1/02

(52) U.S. Cl. 435/6; 435/29

(57) ABSTRACT

The present invention is directed to methods for identifying a specific class of competitive inhibitors of HIV reverse transcriptase that act differently from known reverse transcriptase inhibitors.

### FIELD OF THE INVENTION

The present invention is directed to methods for identifying a specific class of competitive inhibitors of HIV reverse transcriptase that act differently from known reverse transcriptase inhibitors.

### BACKGROUND OF THE INVENTION

Drugs that are currently on the market or under development to combat HIV viral infection belong to classes such as reverse transcriptase inhibitors (RTIs), protease inhibitors (PIs) and the more recent fusion inhibitors. RTIs prevent viral replication by intervening in the reverse transcription mechanism while PIs intervene in the viral assembly. RT inhibitors interact with the RT enzyme in a number of ways to inhibit its functioning so that viral replication becomes blocked. PIs bind to the active site of the viral protease enzyme, thereby inhibiting the cleavage of precursor poly proteins necessary to produce the structural and enzymatic components of infectious virons.

Nucleoside Reverse Transcriptase Inhibitors (NRTIs) are a class of RT inhibitors that are intracellularly converted to nucleoside triphosphates that compete with the natural nucleoside triphosphates for incorporation into elongating viral DNA by reverse transcriptase. Chemical modifications that distinguish these compounds from natural nucleosides result in DNA chain termination events. NRTIs that are currently available include zidovudine (AZT), didanosine (ddI), zalcitabine (ddC), stavudine (d4T), lamivudine (3TC), abacavir (ABC), emtricitabine (FTC) and tenofovir and tenofovir disoproxil fumarate (TDF), the latter often being referred to as Nucleotide Reverse Transcriptase Inhibitors (NtRTIs).

The function of the reverse transcriptase enzyme is to convert the RNA of HIV into DNA. In this process deoxynucleoside triphosphates (dNTP) are coupled to the growing DNA chain. Chain stoppers such as the NRTIs are first converted to the triphosphate (TP) by cellular kinases. For example AZT, which was one of the first HIV RT inhibitors identified, is converted to AZT-TP and HIV-1 RT is subsequently able to use AZT-TP as an efficient alternative substrate in the building of the viral DNA. However, AZT-TP lacks a 3′OH necessary for further DNA elongation, thereby causing termination of the growing DNA chain following incorporation. The inverse process, namely the removal of chain nucleotides or the removal of the chain-terminating residue such as AZT, is mediated by pyrophosphate or nucleoside triphosphates, also takes place but to a far lesser extend. This inverse process is enhanced in HIV mutants, which have increased capability to remove the chain-terminating residue with much greater efficiency than wild type RT. This mechanism is seen as the cause of resistance of mutated HIV against AZT or any of the other NRTIs as described, for example in Gotte et al., Journal of Virology, 2000, pp. 3579-3585.

Because of this inverse process, the addition of pyrophosphate or nucleoside triphosphates to an in vitro RT test model results in reduced inhibitory activity of the NRTI tested, in particular when mutated RT is used. Some NRTIs, however, show only a minimal reduction of RT activity or with some NRTIs RT activity even stays at the same level.

Resistance of the HIV virus against currently available HIV drugs continues to be a major cause of therapy failure. This has led to the introduction of combination therapy of two or more anti-HIV agents usually having a different activity profile. Significant progress was made by the introduction of HAART therapy (Highly Active Anti-Retroviral Therapy), which has resulted in a significant reduction of morbidity and mortality in HIV patient populations treated therewith. HAART involves various combinations of NRTIs, NNRTIs and PIs. Current guidelines for antiretroviral therapy recommend such triple combination therapy regimen for initial treatment. However, these multidrug therapies do not completely eliminate HIV and long-term treatment usually results in multidrug resistance. In particular, half of the patients receiving anti-HIV combination therapy do not respond fully to the treatment, mainly because of resistance of the virus to one or more drugs used. It also has been shown that resistant virus is carried over to newly infected individuals, resulting in severely limited therapy options for these drug-naive patients.

Consequently, there is a continued need for new types of active ingredients for use in drug combinations that are effective against HIV. Providing new types of anti-HIV effective active ingredients, differing in chemical structure and activity profile therefore is a highly desirable goal to achieve. Reverse transcriptase remains an interesting target and inhibitors of this enzyme are an indispensable part of HAART combinations. Finding agents that block the functioning of this enzyme via a new mechanism are expected to provide an alternative to the currently used NRTIs and NNRTIs. In particular the latter suffer from mutations that cause cross-resistance along this whole class. But also the NRTIs, although to a lesser extend, face resistance due to mutations. The fact that NRTIs show less cross-resistance is explained by a more complicated interaction with RT compared to the NNRTIs which apparently all interact with the same binding pocket so that a mutation causing a structural change in this pocket results in all NNRTIs becoming ineffective. Therefore, finding compounds that have an NRTI like behavior but are chemically different, is expected to result in drugs that are less sensitive to cross-resistance and that select different mutations. Such compounds could find use as alternatives for NNRTIs and/or NRTIs in drug cocktails.

The present invention is aimed at methods to identify HIV RT inhibitors that belong to a new class in that they interact differently with reverse transcriptase compared to the currently known NRTIs or NNRTIs. Compounds belonging to this new class of RT inhibitors do not belong to the current classes of NRTIs nor of NNRTIs and therefore may find use as alternatives for drugs belonging to these classes and in particular they may find use in anti-HIV drug combinations.

It additionally has been found that some RT inhibitors unexpectedly show increased activity when a pyrophosphate or a nucleoside triphosphate is added in an in vitro test model. As mentioned above, only a decrease or a staying at the same level of RT activity has been found so far. Consequently compounds showing such increased activity are believed to interact differently with the RT enzyme and therefore belong to a new class of RT inhibitors. The present invention additionally is aimed at detecting compounds that not only are competitive towards the incorporated nucleotide but also show increased activity when a pyrophosphate or a nucleoside triphosphate is added.

### SUMMARY OF THE INVENTION

The present invention provides a method for identifying a new class of nucleotide competitive RT inhibitors comprising:
a) providing test compounds that are other than nucleoside triphosphates;
b) subjecting the said test compounds to a wild-type HIV virus replication test in cells;
c) subjecting test compounds to a NNRTI resistant HIV virus replication test in cells;
d) subjecting the test compounds to a kinetic reverse transcriptase enzymatic assay; and identifying the test compounds that are competitive towards the incorporated nucleotide in said assay;
   selecting the test compounds that are as well active in step b), are active in step c) and are identified as being competitive in step d).

In a further aspect, the present invention provides a method for identifying a new class of ribonucleotide or pyrophosphate sensitive and nucleotide competitive RT inhibitors comprising:
a) providing test compounds that are other than nucleoside triphosphates;
b) subjecting the said test compounds to a wild-type HIV virus replication test in cells;
c) subjecting test compounds to a NNRTI resistant HIV virus replication test in cells;
d) subjecting the test compounds to a kinetic reverse transcriptase enzymatic assay; and identifying the test compounds that are competitive in said assay;
e) selecting the test compounds that are as well active in step b), are active in step c) and are identified as being competitive in step d);
f) providing a reaction well comprising at least one template for an HIV RT enzyme,
   at least one primer,
   at least one detectable dNTP substrate,
   at least one test compound;
   at least one RT enzyme, wherein said HIV RT enzyme incorporates the detectable dNTP substrate; and
   determining RT activity by measuring the amount of the detectable dNTP substrate incorporated into the template;
g) providing another reaction well comprising
   at least one template for an HIV RT enzyme,
   at least one primer,
   at least one detectable dNTP substrate,
   at least one test compound;
   at least one nucleoside phosphate or at least one pyrophosphate,
   at least one RT enzyme, wherein said HIV RT enzyme incorporates the detectable dNTP substrate; and
   determining RT activity by measuring the amount of the detectable dNTP substrate incorporated into the template;
h) comparing the RT activity obtained in step f) and in step g)
i) selecting the test compounds that meet the criteria of step e) and wherein the RT inhibitory activity obtained in g) exceeds the RT inhibitory activity obtained in f); wherein the amount of the HIV RT inhibitor in steps f) and g) is the same and is such that an increase of RT activity is measurable.

The present invention additionally is directed to various combinations and sub-combinations of any of the features disclosed in this specification and claims.

### DETAILED DESCRIPTION OF THE INVENTION

The selection of the anti-HIV compounds as in step b) of the method of this invention may be done using an in vivo assay with wild-type HIV virus. Inhibition of replication of the virus can be determined by measuring the EC values which are the concentrations of the test compounds that are required to protect a certain percentage of the cells from becoming infected with HIV virus. EC₅₀ or EC₉₀ values can be determined which are the concentrations of the test compounds that are required to protect a 50% or 90%, respectively, of the cells from becoming infected with HIV virus. EC values at other percentages may be determined but usually EC₉₀ and in particular EC₅₀ values are preferred. These values may be measured using known procedures.

One such procedure is based on cells, which can send a detectable signal when infected with HIV. Cells for use in this procedure preferably are highly susceptible to and permissive for HIV infection. The detectable signal can be any signal used in biochemical or biological tests such as radioactive labeling, fluorescence, luminescence, or absorption spectrometry. Detectable signals also include certain events in the cell such as cell necrosis or any other signal associated with HIV infection of the cell. The detectable signals can be measured directly or indirectly. In certain embodiments the cell have been engineered to send a detectable signal when infected with HIV.

In one embodiment the cells are engineered with GFP and an HIV-specific promoter and ongoing HIV-infection can be measured fluorometrically. Cytotoxicity is measured in the same cells, but engineered with GFP under a constitutional promotor. The infection (or inhibition thereof) of HIV infected cells and the fluorescence of mock-infected cells is assessed by the fluorescent GFP signal generated by the two above mentioned types of cells. Cells that can be used include HIV-1 transformed T4-cells, MT-4, which were previously shown (Koyanagi et al., Int. J Cancer, 36, 445-451, 1985) to be highly susceptible to and permissive for HIV infection, serving as the target cell line. In these cells, engineered with GFP (and an HIV-specific promotor), ongoing HIV-infection is measured fluorometrically. This embodiment provides a rapid, sensitive and automated assay procedure that can be used for the in vitro evaluation of anti-HIV agents.

Effective concentration values such as 50% effective concentration (EC₅₀) can be determined and are usually expressed in μM. An EC₅₀ value then is defined as the concentration of test compound that reduces the fluorescence of HIV-infected cells by 50%. The 50% cytotoxic concentration (CC₅₀ in μM) is defined as the concentration of test compound that reduces fluorescence of the mock-infected cells by 50%. The ratio of CC₅₀ to EC₅₀ is defined as the selectivity index (SI). Measurements are done before cell necrosis, which usually takes place about five days after infection, in particular measurements are performed three days after infection.

In another embodiment, the measurement is based on the cytopathogenic effect of the HIV virus. Effective concentration values such as 50% effective concentration (EC₅₀) or the 90% effective concentration (EC₉₀) represent the amount of test compound required to protect a certain percentage such as 50% or 90% of the cells from the cytopathogenic effect of the virus. Preferably EC₅₀ values are used.

In this procedure, HIV- or mock-infected MT4 cells are incubated for a number of days, usually five days, in the presence of various concentrations of the test compound. At the end of the incubation period, all HIV-infected cells are killed by the replicating virus in the control cultures, in the absence of any inhibitory test compound. Cell viability is measured by standard techniques, e.g. by measuring the concentration of MTT, a yellow, water soluble tetrazolium dye that is converted to a purple, water insoluble formazan in the mitochondria of living cells only. Upon solubilization of the resulting formazan crystals with isopropanol, the absorbance of the solution is monitored at 540 nm. The values correlate directly to the number of living cells remaining in the culture at the completion of the five day incubation. The inhibitory activity of the compound was monitored on the virus-infected cells and was expressed as EC₅₀ or EC₉₀ values. These values represent the amount of the compound required to protect 50% and 90%, respectively, of the cells from the cytopathogenic effect of the virus. The toxicity of the test compound is measured on the mock-infected cells and is expressed as CC₅₀, which represents the concentration of test compound required to inhibit the growth of the cells by 50%. The selectivity index (SI) (ratio CC₅₀/EC₅₀) can also be determined and is an indication of the selectivity of the anti-HIV activity of the test compound. Results can also be reported as e.g. pEC₅₀ or pCC₅₀ values, the negative logarithm of the result expressed as EC₅₀ or EC₉₀ respectively.

The test results obtained in step b) are evaluated to see if the test compounds are effective in inhibiting wild-type HIV virus replication. Effectiveness of a test compound in inhibiting wild-type HIV virus replication may be evaluated by determining the EC₅₀ value of a given test compound to be smaller than a given EC₅₀ value which is deemed to be a threshold value for effectiveness. This threshold value may be positioned at about 100 μM (pEC₅₀ is about 4), preferably it may be positioned at about 32 μM (pEC₅₀ is about 4.5).

Wild type virus can be of various sources, e.g. LAI strain or HXB2 strain.

The selection of the anti-HIV compounds as in step c) of the method of this invention is done using the same type of assays as in step b) but using mutant HIV virus that is resistant towards NNRTIs. As in step b), inhibition of replication of the virus can be determined by measuring the EC values which are the concentrations of the test compounds that are required to protect a certain percentage of the cells from becoming infected with HIV virus. EC₅₀ or EC₉₀ values can be determined which are the concentrations of the test compounds that are required to protect a 50% or 90%, respectively, of the cells from becoming infected with HIV virus. EC values at other percentages may be determined but usually EC₉₀ and in particular EC₅₀ values are preferred. These values may be measured using known procedures, in particular the procedures as described in respect to step b).

Mutant HIV strains that can be used are HIV strains that are resistant to NNRTIs. In general this means that the effective concentrations of a test compound which is an NNRTI should be increased when testing the HIV inhibitory effect in mutant HIV infected cells when compared to the same test with wild type infected cells. Preferably, the ratio of the effective concentration of a test compound which is an NNRTI, when testing the HIV inhibitory effect in mutant HIV infected cells to the effective concentration of the same test in the same test with wild type infected cells, is greater than 1, in particular is about equal or greater than about 4, preferably equal or greater than about 10. The said effective concentrations or EC values can be expressed as EC values at a certain percentage, in particular as EC₅₀ or EC₉₀ values. Preferred are EC₅₀ values.

As used herein, the term ‘NNRTIs’ refers to the group of non-nucleoside reverse transcriptase inhibitors known in the art comprising, but not being limited to nevirapine, delavirdine, efavirenz, 8 and 9-Cl TIBO (tivirapine), loviride, TMC-125, 4-[[4-[[4-(2-cyanoethenyl)-2,6-diphenyl]amino]-2-pyrimidinyl]amino]-benzonitrile (TMC278), dapivirine (R147681 or TMC120), MKC-442, UC 781, UC 782, Capravirine, QM96521, GW420867X, DPC961, DPC963, DPC082, DPC083, calanolide A, SJ-3366, TSAO, 4″-deaminated TSAO, MV150, MV026048, PNU-142721.

Examples of NNRTI resistant HIV strains comprise but are not limited to HIV strains harbouring one or more of the mutations listed in Table 1. These mutations are associated with resistance to NN-reverse transcriptase inhibitors and result in viruses that show resistance to the currently known NNRTIs.

Table 2 lists mutations that may occur additionally to those mentioned in Table 1. These mutations in themselves do not cause resistance to NNRTIs, but are known to strengthen the effect of the mutations of Table 1. Table 3 list multiple mutations which when occurring in HIV, give rise to strong resistance. The mutated strains may be clinically isolated virus strains or site-mutated virus strains (i.e. wild type strains in which the mutation or mutations have been introduced).

TABLE 1

G190A
G190E
G190Q
K101E
K101L
K101P
K101T
K101W
K103H
K103N
K103S
K103T
L100I
M230L
M230I
M230V
V106A
V106M
Y181C
Y181I
Y181V
Y188L
Y188C
Y318F

TABLE 2

| | | |
|---|---|---|
| A98G | V179F | F227C |
| K101Q | Y188H | P236L |
| V108I | G190S | K238N |
| V179I | P225H | K238T |

TABLE 3

| | |
|---|---|
| K101P V179I Y181C | K103N V179I Y181C |
| K103N G190A | K103N Y181C |
| K103N V179I | K103N Y181C |
| K103N V179I G190A | K103N Y181C E194G |
| K103N Y181C Y318F | V106A Y181C |
| K103N Y318F | V179F Y181C |
| L100I K103N | V179F Y181C |
| L100I K103N | V179I Y181C |
| L100I K103N T386A | V179I Y181C G190A |
| L100I K103N V179I | V179I Y188H G190A |
| L100I K103N Y181C | Y181C G190A |
| V106A F227L | Y181C Y318F |

Preferred for use in step c) of the methods of the invention are virus strains with the mutations listed in Table 3. Of particular interest is the strain that contains the K103N and Y181C mutation.

The kinetic enzymatic assay as in step d) is used to determine whether or not a test compound is a competitive RT inhibitor and is an enzymatic kinetics assay in which the mechanism of inhibition of the HIV RT inhibitor is determined from the kinetics using a wild type HIV RT or mutant RT protein. For example, the Michaelis constant, Km, the dissociation constant of the enzyme-inhibitor complex, Ki, and the mechanism of inhibition may be determined by fitting the data at various concentrations of substrate, RT inhibitor, and/or other reagents, to the Michaelis-Menten competitive inhibition equation, the Michaelis-Menten non-competitive inhibition equation and the Michaelis-Menten un-competitive inhibition equation. If the best fit is obtained using the Michaelis-Menten competitive inhibition equation, than the inhibitor is a nucleotide-competitive RT inhibitor. Any other kinetic analysis known in the art may be utilized with the methods of the invention depending on the application envisaged.

Steps b), c) and d) may be conducted in any given sequence, they may be conducted sequentially or in parallel. When conducted sequentially, step b) may be conducted first followed by step c) and then step d) or vice versa. Two or all three of the steps may be run in parallel while the other step is run before or after the running of both steps. In a preferred execution, first step b) is conducted, followed by step c) and followed by step d).

In order to streamline the testing, the compounds which in are active in step b) are selected and run in test c) whereafter only the compounds that are active in test c) are run in test d). A preferred embodiment of the invention therefore is a method for identifying a new class of nucleotide competitive RT inhibitors comprising:
a) providing test compounds that are other than nucleoside triphosphates;
b) selecting anti-HIV compounds which inhibit replication of wild-type HIV virus;
c) testing the compounds selected in step a) against NNRTI resistant virus strains and selecting those compounds which inhibit replication of said virus strains;
d) subjecting the compounds selected in b) to a kinetic enzymatic assay and selecting the compounds that are competitive in said assay.

Compounds, which are nucleoside triphosphates should be discarded as specified in step a) of the methods of the invention. These comprise any of the triphosphates of natural nucleosides or of derivatives thereof such as the NRTI triphosphates, in particular these comprise any triphosphates that compete with the natural nucleoside triphosphates for incorporation into elongating viral DNA by reverse transcriptase. In one embodiment, any nucleoside phosphate (nucleotide), either of natural nucleosides or of derivatives thereof, including mono-, di- or triphosphates, is excluded in step a) in the methods of the invention

The invention provides an in vitro test, which is a fast, straightforward and inexpensive method for detecting HIV RT inhibitors belonging to a new class. In a further aspect, the invention provides a method for identifying still a further class of RT inhibitors which can be designated as ‘ribonucleotide or pyrophosphate sensitive and nucleotide competitive’ RT inhibitors, said method comprising steps a)-i) specified above.

In this method the nucleoside phosphate includes ribonucleoside phosphates, ribonucleoside diphosphates and ribonucleoside monophosphates and may also include deoxyribonucleoside triphosphates, deoxyribonucleoside diphosphates and deoxyribonucleoside monophosphates as well as derivatives thereof such as the phosphates of ribonucleoside thio or imino derivatives. The ribonucleoside triphosphates may be chosen from ATP, GTP, UTP, CTP, the deoxyribonucleoside triphosphates may be chosen from dATP, dGTP, dUTP, TTP, dCTP. The ribonucleoside mono or diphosphates may be chosen from AMP, ADP, GMP, GDP, UMP, UDP, CMP, CDP. The deoxyribonucleoside mono or diphosphates may be chosen from dAMP, dADP, dGMP, dGDP, dUMP, dUDP, TDP, TMP, dCMP, dCDP. The phosphates of ribonucleoside thio or imino derivatives include for example ATPbgNH (adenosine 5′(beta, gamma, imido)triphosphate), ATPgS (adenosine 5′[gamma-thio]triphosphate). Of particular interest are ADP, AMP, ATPbgNH, ATPgS, ATP, CTP, GTP, UTP.

Where the nucleoside phosphate is a deoxyribonucleoside triphosphate, the detectable dNTP substrate preferably is derived form another nucleic acid. Preferred for use in the invention are ATP or GTP, most preferred is ATP. The pyrophosphate or PPi may be a pyrophosphate salt such as an alkalimetal pyrophosphate, in particular sodium pyrophosphate.

In the methods of this invention, the ingredients of step b) and of step c) may be added to the test well in any given sequence. They may be added one by one or group wise such as combined in a mixture. In particular, the RT enzyme may be added first to the reaction well and then the other components are added or the other components may be added first, where after the RT enzyme is added to the reaction well. Also possible is that one or more of the components are added and then the RT enzyme, followed by the remaining components.

Thus in one embodiment, the assay provides a reaction well comprising a template for an HIV RT enzyme, a primer, a detectable dNTP substrate and a test compound. A HIV RT enzyme is then added to the reaction well, wherein the HIV RT enzyme incorporates the detectable dNTP substrate into the template. RT inhibitory activity of the test compound is measured. The test compound is subjected to another test in which another reaction well is provided comprising a template for an HIV RT enzyme, a primer, a detectable dNTP substrate, the test compound and a nucleoside phosphate, such as ATP and GTP, or a pyrophosphate. A wild type HIV RT enzyme is then added to the reaction well, wherein the HIV RT enzyme incorporates the detectable dNTP substrate into the template. RT inhibitory activity of the test compound in the presence of the nucleoside phosphate or the pyrophosphate is measured and compared with that obtained in the test without the nucleoside phosphate or the pyrophosphate. Those compounds are selected wherein the RT inhibitory activity of the test compound in the presence of the nucleoside phosphate or the pyrophosphate exceeds the RT inhibitory activity obtained in the test without the nucleoside phosphate or the pyrophosphate.

In another embodiment of the invention, the assay is conducted such that the HIV RT enzyme is present in a reaction well and the template, primer, detectable dNTP substrate, HIV RT inhibitor, and, in the second part of the assay the nucleoside phosphate or the pyrophosphate, are added to the HIV RT enzyme.

In the methods including steps f), g), h) and i), steps b), c), d), f) and g) may be conducted in parallel or sequentially, meaning that any combination of parallel or sequential runs can be done. For example all five steps can be run in parallel or all five steps can be run sequentially in any given sequence, i.e. some steps in parallel and other sequentially, e.g. step b), c) and d) sequentially, followed or preceded by steps f) and g) in parallel etc.

The methods including steps f), g), h) and i), are based on a change in susceptibility of the RT enzymatic activity to a certain test compound. Susceptibilities can be generally expressed as ratios of IC₅₀ or IC₉₀ values of RT enzymatic activity in the presence and in the absence of a nucleoside phosphate, such as ATP or GTP, or a pyrophosphate. Ratios of other IC percentages are also possible. Susceptibilities are generally expressed as ratios of IC₅₀ or IC₉₀ values.

The IC₅₀ or IC₉₀ value is the test compound concentration at which 50% or 90% respectively of the enzymatic activity is inhibited. These values are determined using standard procedures.

The ratio of the IC value of RT enzymatic activity measured in the presence a nucleoside phosphate, e.g. ATP and GTP, or a pyrophosphate to the IC value of RT enzymatic activity measured in the presence a nucleoside phosphate, e.g. chosen from ATP and GTP, or a pyrophosphate should greater than 1, preferably said ratio should be greater than 3, more preferably it should be greater than 5. In particular said ratio is the ratio of the IC₅₀ or IC₉₀ values.

The invention provides an in vitro, fast, and inexpensive method for detecting HIV RT inhibitors belonging to a new class.

The methods of the invention are especially applicable in high throughput testing or evaluation devices. It is within the practice of the invention, however, to prepare a sample rack or solid support made up of numerous reaction wells, such that each reaction remains isolated form one another. Simultaneous transfer of one or more reagents to the reaction wells may then be achieved by one of the many techniques used in the art of high throughput analysis.

In the methods including steps e), f), g) and h), one or more of the contents of each of the reaction wells may be varied. For example, in one embodiment, each reaction well contains a template for an HIV RT inhibitor, a primer, a detectable dNTP substrate and a HIV RT enzyme which can be wild-type RT enzyme or a mutant RT enzyme. A nucleoside phosphate, e.g. chosen from ATP or GTP of a pyrophosphate is or is not added to each reaction well. The reaction wells may form an array or may employ another means of identifying or addressing each compartment. The RT activity of each reaction well may then be automatically determined from the amount of detectable dNTP substrate incorporated into the template of each reaction well, and recorded. Other embodiments include, but are not limited to varying the concentration of one or more of the components, the RT enzyme, and varying the nucleoside phosphate or pyrophosphate.

There are numerous methods for handling high throughput. i.e., analyzing a large number of samples in a relatively short period of time. Any method of high throughput analysis available may be applied to the methods of the invention. Examples include, but are not limited to: U.S. Pat. No. 5,985,215 of Sakazume et al., entitled ‘Analyzing Apparatus Having a Function Pipette Samples’; U.S. Pat. No. 6,046,056 of Parce et al., entitled ‘High Throughput Screening Assay Systems in Microscale Fluidic Devices’; WO 00/14540 of Pauwels et al., entitled ‘Method For the Rapid Screening of Analytes’; WO 99/30154 of Beutel et al., entitled ‘Continuous Format High Throughput Screening’; and WO 99/67639 of Wada et al., entitled ‘High Throughput Methods, Systems and Apparatus for Performing Cell Based Screening Assays’.

In the practice of the invention, any template that would serve as an effective template for an HIV RT enzyme may be used. The template may or may not be bound to the reaction well, but in a preferred embodiment is bound to the reaction well. In a further preferred embodiment the template is chosen from poly-rA or a heteropolymer RNA or DNA. Any primer complementary to the template chosen may be used. In one embodiment, the primers are chosen from oligo-dT or a primer complementary to the heteropolymer template.

Detectable dNTP substrates useful in the practice of the invention include any dNTP substrate, and in a preferred embodiment, any dTTP substrate (deoxythymidine triphosphate), that is detectable before and/or after integration into the template. Detectable dNTP substrates include but are not limited to a radioactive labeled dTTP or any radioactive labeled dNTP, and a dNTP substrate that is capable of being detected by fluorescence, luminescence, or absorption spectrometry. The detectable dNTP substrate may be detectable on its own or it may bind to a tracer, which may then be detected. The tracer may be an optical tracer, such as a tracer that may be detected by fluorescence, luminescence, or absorption spectrometry, or the tracer may be a radioactive labeled tracer. In one embodiment, the detectable dNTP substrate is bromo-deoxyuridine-triphosphate and the optical tracer is an antibody or a monoclonal antibody such as monoclonal anti-BrdU antibody, conjugated to alkaline phosphatase that binds to the dNTP substrate.

The test compounds for use in the methods of this invention can be any natural, natural derived or man-made materials. As used in this context the term ‘test compound’ refers to single compounds or to mixtures of compounds.

In the methods including steps e), f), g) and h), the reaction wells for use in the methods of the invention may or may not contain at least one nucleoside phosphate, e.g. chosen from ATP and GTP, or at least one pyrophosphate. The concentrations of ribonucleotides or pyrophosphates may be varied depending on the application envisaged. For example, the influence of pyrophosphate (together with ATP) in vivo obviously depends on the intracellular concentrations. In a preferred embodiment, the intracellular concentrations, which are well established at 3.2±1.5 mM for ATP, 0.5±0.2 mM for GTP, and 130 μM for pyrophosphate are used. The methods of the invention may be initiated by using wild-type RT enzyme or mutant RT enzyme. Any RT enzyme or mutant RT enzyme that may incorporate the detectable dNTP substrate into the template may be useful in the practice of the invention.

The invention also provides for a kit. The kit may be used for any of the methods described herein, in particular the kit may be used to select nucleotide-competitive RT inhibitors. The kit of the invention may comprise a template for an HIV RT enzyme; a primer; a detectable dNTP substrate; and a ribonucleoside triphosphate, e.g. chosen from ATP and GTP, or a pyrophosphate. The kit may further comprise a mutant RT enzyme and/or a wild type RT enzyme.

In the methods including steps e), f), g) and h), The methods of the invention may be conducted using wild-type RT enzyme or mutant RT enzyme. Any RT enzyme or mutant RT enzyme that may incorporate the detectable dNTP substrate into the template may be useful in the practice of the invention.

The methods of the invention may find use in high throughput testing or evaluation devices. It is within the practice of the invention, however, to prepare a sample rack or solid support made up of numerous reaction wells, such that each reaction remains isolated form one another. Simultaneous transfer of one or more reagents to the reaction wells may then be achieved by one of the many techniques used in the art of high throughput analysis.

The test compounds for use in the methods of this invention can be any natural, natural derived or man-made materials. As used in this context the term ‘test compound’ refers to a single compound or to a mixture of compounds.

The methods according to the present invention allow the detection of compounds that are RT inhibitors, which belong to a new class, which are referred to as “nucleotide competitive RT inhibitors (NCRTIs)” that do not belong to the classes of NRTIs nor of NNRTIs. The latter term is used in the art to comprise those compounds, which interact with the so-called NNRTI ‘pocket’ in the RT enzyme. All current NNRTIs have been found to bind in the same hydrophobic pocket, which is believed to cause the relative quick emergence of inactivating mutations as well as cross-resistance all over this class of HIV inhibitors. But also the NRTIs, although to a lesser extend, face resistance due to mutations. The fact that NRTIs show less cross-resistance is explained by a more complicated interaction with RT compared to the NNRTIs.

Compounds of this new class are unique in that they are structurally different from the class of NRTIs but nevertheless show NRTI-like behavior in that they compete with the natural nucleoside triphosphates. They may be used as alternative treatment in patients infected with HIV mutants that escape NRTIs and NNRTIs or may find use in anti-HIV drug combinations.

The methods including steps h), g), h) and i) may be used to detect HIV inhibitors belonging to another class, namely nucleotide competitive RT inhibitors that do not belong to the classes of NRTIs nor of NNRTIs and moreover show increased RT inhibitory activity in the presence of a nucleoside phosphate or a pyrophosphate. Without wishing to be bound by the following theory, it is assumed that such compounds interact at the same location in the RT enzyme as the NRTIs, while the nucleoside phosphate (such as ATP) or the pyrophosphate also bind to the RT enzyme in such way that the functioning of the enzyme is blocked. This results in a double blocking of the enzyme so that compounds detected by the methods of this invention are believed to be very effective blockers of the HIV RT enzyme. Even without co-administration of a nucleoside phosphate or of a pyrophosphate this effect may play a role because nucleoside phosphates such as ATP are present in cell plasma.

The invention further provides methods for screening of compounds in order to identify the compounds that belong to the new class of HIV RT inhibitors described herein.

All references, patents, and patent applications cited herein are incorporated by reference in their entirety.

The following examples are provided by way of illustration and are not intended to be limiting of the present invention.

### EXAMPLES

### Example 1

The test compounds are examined for anti-viral activity in a cellular assay performed according to the following procedure.

HIV- or mock-infected MT4 cells were incubated for five days in the presence of various concentrations of the test compound. At the end of the incubation period, the replicating virus in the control cultures had killed all HIV-infected cells in the absence of any inhibitor. Cell viability was determined by measuring the concentration of MTT, a yellow, water soluble tetrazolium dye that is converted to a purple, water insoluble formazan in the mitochondria of living cells only. Upon solubilization of the resulting formazan crystals with isopropanol, the absorbance of the solution was monitored at 540 nm. The values correlate directly to the number of living cells remaining in the culture at the completion of the five day incubation. The inhibitory activity of the compound was monitored on the virus-infected cells and was expressed as EC₅₀ and EC₉₀. These values represent the amount of the compound required to protect 50% and 90%, respectively, of the cells from the cytopathogenic effect of the virus. The toxicity of the compound was measured on the mock-infected cells and was expressed as CC₅₀, which represents the concentration of compound required to inhibit the growth of the cells by 50%. The selectivity index (SI) (ratio CC₅₀/EC₅₀) is an indication of the selectivity of the anti-HIV activity of the inhibitor. Wherever results are reported as e.g. pEC₅₀ or pCC₅₀ values, the result is expressed as the negative logarithm of the result expressed as EC₅₀ or CC₅₀ respectively.

Compound 1 has an EC50 on wild-type HIV-1 (LAI strain) of 45 nM.

Compound 1 has been described in WO-04/046143 is the compound having the structure

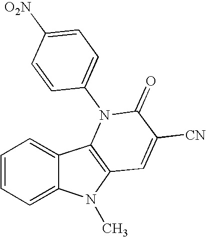

### Example 2

The test compounds were also tested for their potency against HIV strains harbouring several mutations leading to resistance against NNRTI inhibitors (Tables 1 and 7). The test compounds were submitted to the same test procedures as set out in example 1, but replacing the wild type virus by mutated virus that is resistant towards NNRTI inhibitors.

Compound 1 has an EC50 of 98 nM on a site-directed mutant of wild-type HIV-1 (HXB2) that contains the K103N and Y181C mutation.

### Example 3

### Enzyme Kinetics Studies

The enzyme kinetics studies were carried out using a protocol involving a 4×5 matrix of varying substrate and inhibitor concentrations over ranges of 40-3 μM of dTTP and 2-0 μM of compound 1.

The reaction mixtures (50 μl) further contained 50 mM Tris.HCl (pH 7.8), 5 mM dithiothreitol, 300 mM glutathione, 500 μM EDTA, 150 mM KCl, 5 mM MgCl₂, 0.15 mM of the template/primer poly(rA)oligo(dT) and 0.06% Triton X-100.

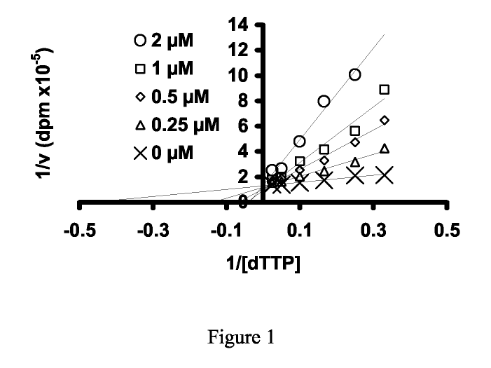

The reaction mixtures were incubated at 37° C. for 15 min, at which time 100 μl of calf thymus DNA (150 pg/ml), 2 ml of Na₄P₂O₇ (0.1 M in 1 M HCl), and 2 ml of trichloroacetic acid (10% v/v) were added. The solutions were kept on ice for 30 min, after which the acid-insoluble material was washed and analyzed for radioactivity. When the reciprocal of the reaction velocity (1/v) is plotted against the reciprocal of the substrate concentration (1/[dTTP]) in the presence of different concentrations of compound 1, the graph (See FIG. 1) is obtained.

Since all the lines cross at the intercept with the Y-axis it is clear that this compound has a competitive mode of HIV RT inhibition.

### Example 4

### In Vitro Inhibition of HIV Reverse Transcriptase in Presence and Absence of ATP

The assay was run using kit TRK 1022™ (Amersham Life Sciences) according to the manufacturer's instructions with slight modifications. Test compounds were diluted in steps of ¼ in 100% DMSO and subsequently diluted 1/50 in Medium A (RPMI 1640+10% fetal calf serum, 20 mg/ml gentamycin).

In each experiment three conditions were tested: wells filled with 25 μl of the above compound solutions, wells filled with 25 μl 2% DMSO in Medium A (RO) and wells filled with 100 μl Stopsolution and 25 μl DMSO in Medium A (RI). To each well was added 25.5 μl master mix (5 μl primer/template beads, 10 μl assay buffer, 0.5 μl tracer (50 μM [3H]-dTTP), 5 μl HIV RT enzyme solution (15 mU/μl), 5 μl Medium A). The plates were sealed, and incubated during 4 hours at 37° C. Subsequently, 100 μl stop solution was added to each well (except RI). The radioactivity was counted in a TopCount™.

For testing compound inhibition in the presence of ATP the same protocol as above was used but the Medium A in the master mix was replaced with Medium A containing 320 mM ATP.

Using the assay above the IC₅₀ of compound 1 in the absence of ATP is 0.3 μM while the IC₅₀ of compound 1 in the presence of ATP is 0.016 μM.

### Example 5

The procedures of example 1 were repeated but ATP was changed by a number of other nucleoside phosphates. The following table lists the tested nucleoside phosphates and the IC₅₀ values in μM of compound 1 obtained in the presence of the concerned nucleoside phosphate.

ATPgS is adenosine 5′[gamma-thio]triphosphate[CAS 93839-89-5]; and

ATPbgNH is adenosine 5′(beta, gamma, imido)triphosphate [CAS 72957-42-7].

| Nucleoside phosphate | IC₅₀ in μM |
|---|---|
| ADP | 0.023 |
| AMP | 0.0178 |
| ATPbgNH | 0.0245 |
| ATPgS | 0.0124 |
| CTP | 0.114 |
| GTP | 0.0704 |
| UTP | 0.0519 |
| PPi | 0.1992 |

1. A method for identifying a new class of nucleotide competitive RT inhibitors comprising:
a) providing test compounds that are other than nucleoside triphosphates;
b) subjecting test compounds to a wild-type HIV virus replication test in cells;
c) subjecting test compounds to a NNRTI resistant HIV virus replication test in cells;
d) subjecting the test compounds to a kinetic reverse transcriptase enzymatic assay; and identifying the test compounds that are competitive towards the incorporated nucleotide in said assay;
selecting the test compounds that are as well active in step b), are active in step c) and are identified as being competitive towards the incorporated nucleotide in step d).

2. A method according to claim 1 comprising:
a) providing test compounds that are other than nucleoside triphosphates;
b) selecting anti-HIV compounds which inhibit replication of wild-type HIV virus;
c) testing the compounds selected in step b) against NNRTI resistant virus strains and selecting those compounds which inhibit replication of said virus strains;
d) subjecting the compounds selected in c) to a kinetic enzymatic assay and selecting the compounds that are competitive in said assay.

3. A method for identifying a new class of ribonucleotide or pyrophosphate sensitive and nucleotide competitive RT inhibitors comprising:
a) providing test compounds that are other than nucleoside triphosphates;
b) subjecting test compounds to a wild-type HIV virus replication test in cells;
c) subjecting test compounds to a NNRTI resistant HIV virus replication test in cells;
d) subjecting the test compounds to a kinetic reverse transcriptase enzymatic assay; and
identifying the test compounds that are competitive in said assay;
e) selecting the test compounds that are as well active in step b), are active in step c) and are identified as being competitive in step d);
f) providing a reaction well comprising
at least one template for an HIV RT enzyme,
at least one primer,
at least one detectable dNTP substrate,
at least one test compound;
at least one RT enzyme, wherein said HIV RT enzyme incorporates the detectable dNTP substrate; and
determining RT activity by measuring the amount of the detectable dNTP substrate incorporated into the template;
g) providing another reaction well comprising
at least one template for an HIV RT enzyme,
at least one primer,
at least one detectable dNTP substrate,
at least one test compound;
at least one nucleoside phosphate or at least one pyrophosphate,
at least one RT enzyme, wherein said HIV RT enzyme incorporates the detectable dNTP substrate; and
determining RT activity by measuring the amount of the detectable dNTP substrate incorporated into the template;
h) comparing the RT activity obtained in step f) and in step g)
i) selecting the test compounds that meet the criteria of step e) and wherein the RT inhibitory activity obtained in g) exceeds the RT inhibitory activity obtained in f); wherein the amount of the HIV RT inhibitor in steps f) and g) is the same and is such that an increase of RT activity is measurable.

4. The method of claim 1, wherein steps b) and c) are conducted sequentially.

5. The method of claim 1, wherein steps b) and c) are conducted in parallel.

6. The method of claim 3, wherein steps b) and c) are conducted sequentially followed by step f) and g).

7. The method of claim 6 wherein steps f) and g) are conducted in parallel.

8. The method of claim 3 wherein the RT activity determined in steps f) and g) is a certain percent Inhibitory Concentration, in particular an IC₅₀ or IC₉₀ value.

9. The method of claim 3 wherein the nucleoside phosphate is selected from ATP and GTP.
